(19) United States
(12) Patent Application Publication
(10) Pub. No.: US 2026/0355407 A1
(43) Pub. Date: Nov. 12, 2026

(54) NEGATIVE PRESSURE WOUND THERAPY SYSTEM

(71) Applicant: Solventum Intellectual Properties Company, Maplewood, MN (US)

(72) Inventors: Benjamin A. PRATT, Poole (GB); Michele A. WALDNER, San Antonio, TX (US); Jeffrey G. ZINN, San Antonio, TX (US); Shannon C. INGRAM, San Antonio, TX (US)

(21) Appl. No.: 19/132,683

(22) PCT Filed: Dec. 4, 2023
(86) PCT No.: PCT/IB2023/062205

§ 371 Date: May 23, 2025

Related U.S. Application Data
(60) Provisional application No. 63/431,046, filed on Dec. 8, 2022.

(51) Int. Cl. A61M 1/00

(52) U.S. Cl. CPC A61M 1/92; A61M 1/98; A61M 2205/14; A61M 2205/6018

(57) ABSTRACT

A negative pressure wound therapy (NPWT) system for a wound site includes an instillation dock. The instillation dock includes a pump configured to direct an instillation fluid to the wound site. The instillation dock further includes a port configured to receive a first instillation tube-set and facilitate engagement between the pump and the first instillation tube-set. The first instillation tube-set is adapted to transport the instillation fluid. Further, the port is configured such that, during operation, the instillation fluid is fluidly isolated from the port. The NPWT system further includes a therapy unit adapted to be removably coupled with the instillation dock. The therapy unit includes a negative pressure source and an exudate canister. The negative pressure source is configured to provide negative pressure to the wound site.

### CROSS-REFERENCE TO RELATED APPLICATIONS

This application is a U.S. National Stage Entry of PCT International Application No. PCT/IB2023/062205, filed on Dec. 4, 2023, which claims the benefit of priority to U.S. Provisional Application No. 63/431,046, filed on Dec. 8, 2022, which is incorporated herein by reference in its entirety.

### TECHNICAL FIELD

The present disclosure relates generally to a negative pressure wound therapy (NPWT) system for a wound site, and a method of using the NPWT system.

### BACKGROUND

Negative pressure wound therapy (NPWT) systems are embodied as sealed wound-care systems particularly indicated for wounds, such as, chronic persistent wounds and/or complicated wounds. Specifically, for promoting wound healing, a pressure that is reduced relative to the surroundings (commonly referred to as “negative pressure”) is applied to the wound. The negative pressure causes mechanical contraction of the wound and removal of exudates, such as, slough, necrotic tissue, microbial load (e.g., bacteria and biofilms) from the wound, thus promoting formation of granulation tissues and accelerating wound healing. The NPWT system typically includes a therapy unit that is in fluid communication with the wound.

Recent advancements in wound healing involves instillation of topical fluids to the wound in combination with the negative pressure therapy. Accordingly, the NPWT system includes an instillation device for instilling fluids to the wound. Conventional NPWT system including such an instillation device is typically bulky and complicated to use. Further, the NPWT system may also restrict user mobility mainly due to size and complications involved in connecting and disconnecting a fluid source of the instillation device. Overall, conventional NPWT systems including the instillation device may be complicated to use, may be limited by cost and size, and may not be user friendly.

### SUMMARY

Generally, the present disclosure relates to a negative pressure wound therapy (NPWT) system for a wound site, and a method of using the NPWT system.

In a first aspect, the present disclosure provides a negative pressure wound therapy (NPWT) system for a wound site. The NPWT system includes an instillation dock. The instillation dock includes a pump configured to direct an instillation fluid to the wound site. The instillation dock further includes a port configured to receive a first instillation tube-set and facilitate engagement between the pump and the first instillation tube-set. The first instillation tube-set is adapted to transport the instillation fluid. Further, the port is configured such that, during operation, the instillation fluid is fluidly isolated from the port. The NPWT system further includes a therapy unit adapted to be removably coupled with the instillation dock. The therapy unit includes a negative pressure source and an exudate canister. The negative pressure source is configured to provide negative pressure to the wound site.

In a second aspect, the present disclosure provides a method of using the NPWT system of the first aspect. The method includes connecting the exudate canister with the therapy unit. The method further includes fluidly connecting the exudate canister with a second instillation tube-set. The method further includes connecting the first instillation tube-set with the instillation dock. The method further includes connecting the exudate canister and the second instillation tube-set with a wound dressing applied at the wound site. The method further includes connecting the first instillation tube-set with the instillation dock. The method further includes connecting an instillation fluid reservoir with the first instillation tube-set. The method further includes connecting the therapy unit and the exudate canister with the instillation dock. The method further includes connecting the first instillation tube-set with the second instillation tube-set. The method further includes applying at least one of a negative pressure therapy to the wound site via the therapy unit and a fluid instillation therapy to the wound site via the first instillation tube-set.

### BRIEF DESCRIPTION OF THE DRAWINGS

Exemplary embodiments disclosed herein may be more completely understood in consideration of the following detailed description in connection with the following figures. The figures are not necessarily drawn to scale. Like numbers used in the figures refer to like components. However, it will be understood that the use of a number to refer to a component in a given figure is not intended to limit the component in another figure labeled with the same number.

FIG. 1 illustrates a schematic view of a negative pressure wound therapy (NPWT) system according to an embodiment of the present disclosure;

FIG. 2 illustrates a schematic perspective view of the NPWT system of FIG. 1, according to an embodiment of the present disclosure;

FIG. 3 illustrates a schematic perspective view of an instillation dock and a first instillation tube-set of the NPWT system of FIG. 2, according to an embodiment of the present disclosure;

FIG. 4 illustrates a schematic perspective view of the first instillation tube-set of the NPWT system of FIG. 2, according to an embodiment of the present disclosure;

FIG. 5 illustrates an exploded view of the instillation dock, the first instillation tube-set, and a second instillation tube-set of the NPWT system of FIG. 2, according to an embodiment of the present disclosure;

FIG. 6A illustrates a schematic view of a therapy assembly associated with the NPWT system of FIG. 1 in a detached state, according to an embodiment of the present disclosure;

FIG. 6B illustrates a perspective view of the therapy assembly of FIG. 6A, according to an embodiment of the present disclosure;

FIG. 7 illustrates a block diagram of the NPWT system of FIG. 1, according to an embodiment of the present disclosure;

FIG. 8 illustrates a schematic view of a second instillation tube-set associated with the NPWT system of FIG. 1, according to another embodiment of the present disclosure; and

FIG. 9 illustrates a flowchart for a method of using the NPWT system of FIG. 1, according to an embodiment of the present disclosure.

### DETAILED DESCRIPTION

In the following description, reference is made to the accompanying figures that form a part thereof and in which various embodiments are shown by way of illustration. It is to be understood that other embodiments are contemplated and may be made without departing from the scope or spirit of the present disclosure. The following detailed description, therefore, is not to be taken in a limiting sense.

In the following disclosure, the following definitions are adopted.

As recited herein, all numbers should be considered modified by the term “about”. As used herein, “a,” “an,” “the,” “at least one,” and “one or more” are used interchangeably.

As used herein as a modifier to a property or attribute, the term “generally”, unless otherwise specifically defined, means that the property or attribute would be readily recognizable by a person of ordinary skill but without requiring absolute precision or a perfect match (e.g., within +/−20 % for quantifiable properties).

The term “substantially”, unless otherwise specifically defined, means to a high degree of approximation (e.g., within +/−10% for quantifiable properties) but again without requiring absolute precision or a perfect match.

The term “about”, unless otherwise specifically defined, means to a high degree of approximation (e.g., within +/−5% for quantifiable properties) but again without requiring absolute precision or a perfect match.

Terms such as same, equal, uniform, constant, strictly, and the like, are understood to be within the usual tolerances or measuring error applicable to the particular circumstance rather than requiring absolute precision or a perfect match.

As used herein, the terms “first” and “second” are used as identifiers. Therefore, such terms should not be construed as limiting of this disclosure. The terms “first” and “second” when used in conjunction with a feature or an element can be interchanged throughout the embodiments of this disclosure.

As used herein, “at least one of A and B” should be understood to mean “only A, only B, or both A and B”.

Unless specified or limited otherwise, the terms “attached,” “connected,” “coupled”, and variations thereof, are used broadly and encompass both direct physical connections or indirect physical connections between two or more components that are connected together by one or more additional components. For example, a first component may be coupled to a second component by being directly connected together or by being connected by a third component.

As used herein, the terms “layer,” “sheet,” and “dressing,” or variations thereof, are used to describe an article having a thickness that is small relative to its length and width.

As used herein, the term “negative pressure” broadly refers to a pressure lower than a local pressure in a local environment outside of a sealed treatment environment provided by a dressing. In many cases, the local ambient pressure can also be the atmospheric pressure at which a wound site is located. Alternatively, the pressure can be less than a hydrostatic pressure associated with a tissue at the wound site.

As used herein, the term “wounds” can include, for example, chronic, acute, traumatic, subacute, closed surgical wounds or dehiscence wounds, partially thick burns, ulcers (such as, diabetic, compressive, or venous insufficiency ulcers), flaps, and grafts. The wound may also include an open abdomen area of a patient.

As used herein, the term “wound site” may include a tissue site, such as, bone tissue, adipose tissue, muscle tissue, nerve tissue, skin tissue, vascular tissue, connective tissue, cartilage, tendons, or ligaments. The term “wound site” may also refer to an area of a tissue that is not necessarily a wound or defect but may be desired to add or promote additional tissue growth. For example, negative pressure therapy can be used in a particular tissue area to grow additional tissue that can be harvested or transplanted to another tissue site. The wound site may also include an area wherein a surgical incision has been previously performed.

Conventionally, a negative pressure wound therapy (NPWT) system is used to promote healing of wounds. The NPWT system includes a therapy device configured to provide negative pressure therapy by reducing a pressure at a wound site. The therapy device can draw a vacuum at the wound site (relative to atmospheric pressure) by removing exudates, air, and other fluids from the wound site. The fluids removed from the wound site may be collected within an exudate canister.

Recent advancements in wound healing with the NPWT system involves application of an instillation fluid to the wound site to work in combination with the negative pressure therapy. Accordingly, the NPWT system includes an instillation device for instilling the instillation fluid to the wound. The instillation fluid can include, for example, a cleansing fluid, a prescribed fluid, a medicated fluid, an antibiotic fluid, or any other type of fluid which can be delivered to the wound. The instillation device may include an instillation canister for holding the instillation fluid. NPWT systems including such instillation devices are typically bulky and complicated to use. Further, such NPWT systems may also restrict user mobility mainly due to size and complications involved in connecting and disconnecting the instillation canister. Overall, conventional NPWT systems including the instillation device may be complicated to use, may be limited by cost and size, and may not be user friendly.

The present disclosure provides a NPWT system for a wound site. The NPWT system includes an instillation dock. The NPWT system further includes a therapy unit adapted to be removably coupled with the instillation dock. The therapy unit includes a negative pressure source. The NPWT system further includes an exudate canister configured to be removably coupled with the therapy unit. The exudate canister is in fluid communication with the wound site. The NPWT system further includes a first instillation tube-set adapted to be removably coupled with the instillation dock. The first instillation tube-set is in fluid communication with the wound site to direct an instillation fluid towards the wound site.

The NPWT system disclosed herein may be easy to use. Further, the therapy unit and the exudate canister may be used to apply the negative pressure therapy to the wound site even when the therapy unit and the exudate canister are detached from the instillation dock. Specifically, when the user needs to move, the therapy unit and the exudate canister may be easily detached from the instillation dock to improve mobility. Further, as the therapy unit and the exudate canister may be easily attached and/or detached from the instillation dock, the NPWT system may exhibit improved usability.

The NPWT system may also provide notifications when a fluid instillation therapy is required. Based on the notification, each of the first instillation tube-set, an instillation fluid source, and a second instillation tube-set may be easily attached with the instillation dock. The NPWT system of the present disclosure may further monitor efficacy of connections between the instillation dock and other components of the NPWT system, thereby reducing any risk of inaccurate fluid instillation therapy and/or negative pressure therapy. Further, the NPWT system may be cost-effective as well as portable and compact in size. Thus, the NPWT system may be easy to handle by users, such as, patients or medical personnel.

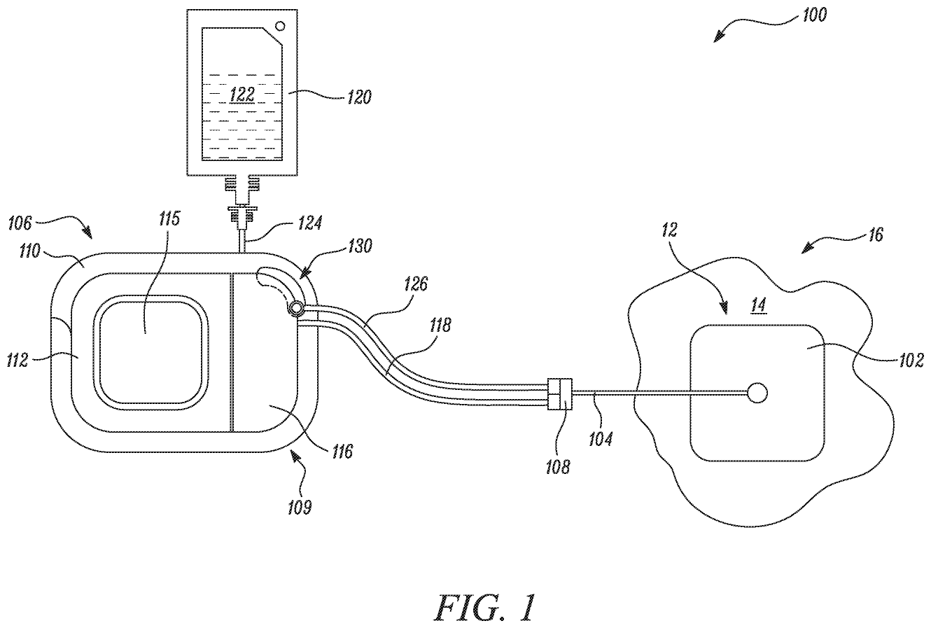

Referring now to Figures, FIG. 1 illustrates a schematic view of a NPWT system 100 for a wound site 12 according to an embodiment of the present disclosure. The NPWT system 100 may be disposed on a skin 14 of a user 16. The user 16 is a patient having a wound (not shown). The skin 14 of the user 16 includes the wound site 12. The NPWT system 100 includes a wound dressing 102 located at the wound site 12. The wound dressing 102 encloses the wound. The NPWT system 100 further includes a dressing tubing 104 connected to the wound dressing 102. The dressing tubing 104 may include two separate fluid passageways (not shown) defined therein. The dressing tubing 104 further includes a splitter 108.

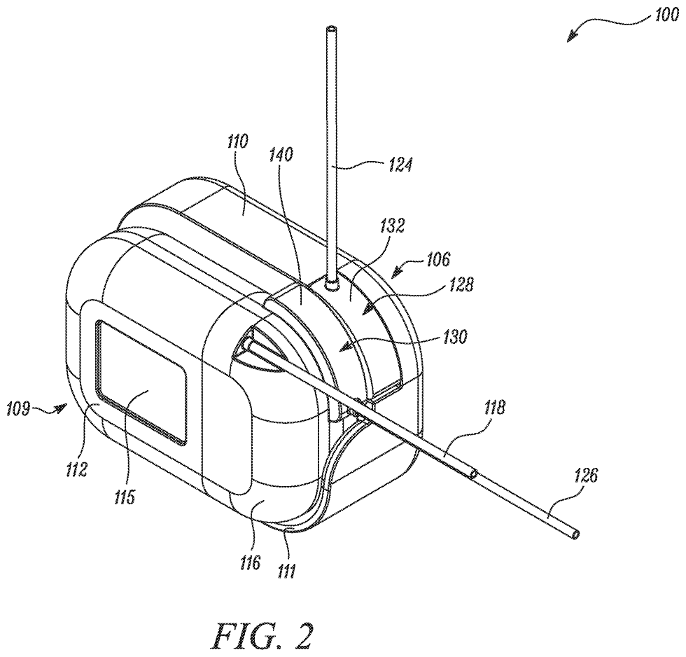

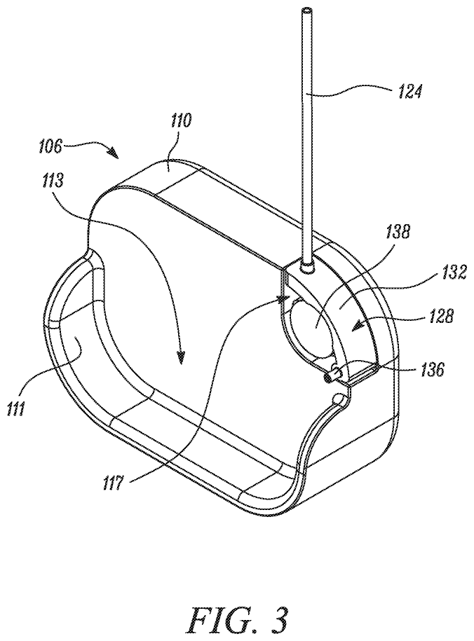

The NPWT system 100 further includes a fluid instillation assembly 106 configured to facilitate a fluid instillation therapy. The NPWT system 100 includes an instillation dock 110. The instillation dock 110 forms a part of the fluid instillation assembly 106. The instillation dock 110 may receive and support one or more components of the NPWT system 100. The instillation dock 110 may include a hollow space (not shown) for holding components, such as, sensors, processing units, alarm indicators, memory/databases, software, display devices, user interfaces, or other devices that facilitate the fluid instillation therapy. The instillation dock 110 is substantially cuboidal. Alternatively, the instillation dock 110 may include any other shape. Further, the instillation dock 110 includes a wall 111 (see FIGS. 2 and 3) and a receptacle area 113 (see FIG. 3) defined by the wall 111.

The NPWT system 100 further includes a therapy assembly 109 configured to apply a negative pressure at the wound site 12. The therapy assembly 109 together with the wound dressing 102 and the dressing tubing 104 may be used to apply a negative pressure therapy at the wound site 12. In some embodiments, the wound dressing 102 and the dressing tubing 104 may form an integral part of the therapy assembly 109. However, the wound dressing 102 and the dressing tubing 104 may be replaceable, as per application requirement. Moreover, the therapy assembly 109 together with the fluid instillation assembly 106, the wound dressing 102, and the dressing tubing 104 may be used to apply the fluid instillation therapy at the wound site 12.

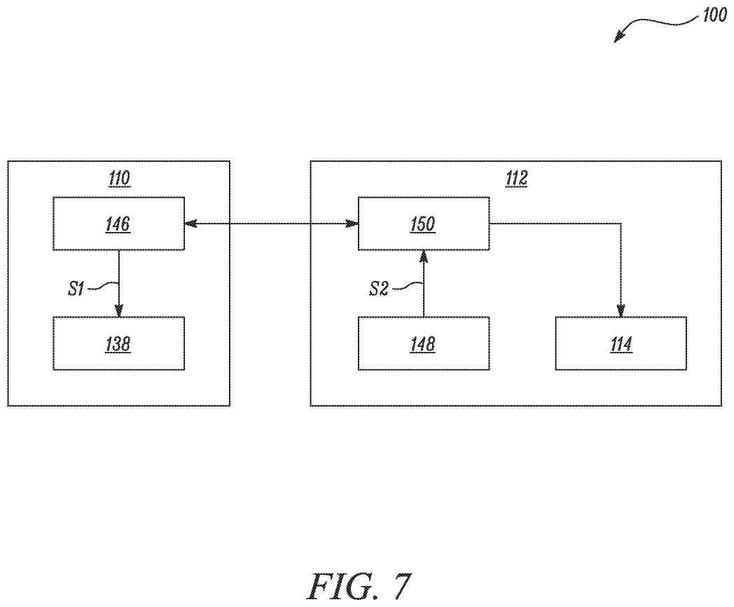

The NPWT system 100 further includes a therapy unit 112 adapted to be removably coupled with the instillation dock 110. Specifically, the therapy unit 112 forms a part of the therapy assembly 109. The therapy unit 112 includes a negative pressure source 114 (schematically shown in FIG. 7) and an exudate canister 116. The negative pressure source 114 is configured to provide negative pressure to the wound site 112. The negative pressure source 114 may be any device which can be operated in order to apply negative or reduced pressure to the wound site 12. The negative pressure source 114 may include an electrically powered device that can reduce pressure in a sealed volume, such as, a vacuum pump, a suction pump, or a micro-pump. The negative pressure source 114 may be housed within the therapy unit 112 and may be used in conjunction with other components, such as, sensors, processing units, alarm indicators, memory/databases, software, display devices, a user interface 115, or other devices that further facilitate negative pressure therapy. The user interface 115 may allow users to provide an input to the therapy unit 112, for example, to initiate the negative pressure therapy or make some adjustments to a negative pressure therapy process. Further, the user interface 115 may also display information related to an ongoing negative pressure therapy or any other information, such as, diagnostics information related to the therapy unit 112. In some embodiments, the user interface 115 may be embodied as a touch screen.

The therapy assembly 109 further includes the exudate canister 116. Negative pressure applied at the wound site 12 can induce macrostrain and microstrain at the wound site 12, as well as remove exudates and other fluids from the wound site 12, which can be collected in the exudate canister 116 and discarded in an appropriate manner. The therapy unit 112 and the exudate canister 116 are accommodated within the receptacle area 113 defined by the instillation dock 110. The exudate canister 116 may include a suitable coupling mechanism that may allow quick coupling of the exudate canister 116 with the therapy unit 112. In an example, the exudate canister 116 may be coupled with the therapy unit 112 via a snap fit, a sliding attachment, and the like. Further, the therapy unit 112 and the exudate canister 116 may be coupled with the instillation dock 110 via a snap fit, a sliding attachment, and the like. Further, the exudate canister 116 may be fluidly coupled to the negative pressure source 114 via a fluid path (not shown). The fluid path may connect the negative pressure source 114 with the exudate canister 116 in a sealing manner.

Further, the exudate canister 116 is in fluid communication with the wound site 12. The exudate canister 116 includes an exudate tube 118. The exudate tube 118 provides fluid communication between the exudate canister 116 and the wound site 12. Specifically, the exudate tube 118 fluidly communicates the exudate canister 116 with the wound site 12, via one of the passageways in the dressing tubing 104. In one exemplary embodiment, the exudate tube 118 may allow the negative pressure generated by the negative pressure source 114 to be delivered to the wound site 12. Further, the exudate tube 118 may also allow removal of exudates from the wound site 12 and collection of exudates in the exudate canister 116.

Further, the fluid instillation assembly 106 includes an instillation fluid reservoir 120 configured to contain an instillation fluid 122 therein. In an example, the instillation fluid reservoir 120 may include a 3M™ V.A.C. Veralink™ Cassette. Further, the instillation fluid 122 may include, for example, a cleansing fluid, a prescribed fluid, a medicated fluid, an antibiotic fluid, or any other type of fluid which can be delivered to the wound site 12 during wound treatment.

As shown in FIGS. 2 and 3, the fluid instillation assembly 106 further includes a first instillation tube-set 128 adapted to be removably coupled with the instillation dock 110. Specifically, the instillation dock 110 includes a port 117 configured to receive the first instillation tube-set 128 and facilitate engagement between a pump 138 and the first instillation tube-set 128. Further, the port 117 is configured such that, during operation, the instillation fluid 122 (see FIG. 1) is fluidly isolated from the port 117. The port 117 may allow a quick coupling of the first instillation tube-set 128 with the pump 138. Further, the first instillation tube-set 128 is adapted to transport the instillation fluid 122. In an example, the first instillation tube-set 128 may be coupled with the instillation dock 110 via a snap fit, without any limitations thereto. The first instillation tube-set 128 is in fluid communication with the wound site 12 (see FIG. 1) to direct the instillation fluid 122 to the wound site 12. The instillation fluid reservoir 120 is in fluid communication with the first instillation tube-set 128.

The NPWT system 100 further includes a second instillation tube-set 130 configured to be coupled with the instillation dock 110. In an example, the second instillation tube-set 130 may be coupled with the first instillation tube-set 128 via a snap fit, without any limitations thereto. Further, the second instillation tube-set 130 is removably coupled to the exudate canister 116. In an example, the second instillation tube-set 130 may be coupled with the exudate canister 116 via a snap fit, without any limitations thereto. The second instillation tube-set 130 is configured to fluidly communicate with the first instillation tube-set 128. The second instillation tube-set 130 is configured to fluidly communicate the first instillation tube-set 128 and the wound site 12. Thus, the instillation fluid reservoir 120 is in fluid communication with the wound site 12 via the first instillation tube-set 128 and the second instillation tube-set 130. In the illustrated embodiment of FIG. 2, the second instillation tube-set 130 is removably disposed between the exudate canister 116 and the first instillation tube-set 128. In another embodiment, the second instillation tube-set 130 may be integral with the exudate canister 116. In yet another embodiment, the second instillation tube-set 130 may be integral with the first instillation tube-set 128.

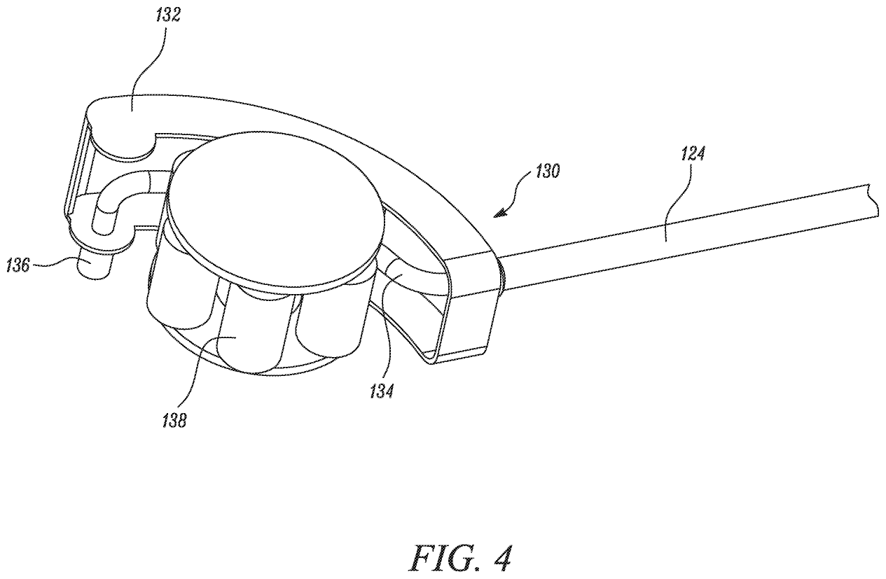

Referring to FIGS. 3 and 4, the first instillation tube-set 128 includes a first carrier 132. The first carrier 132 includes a first fluid tube 134 and a second fluid tube 136. The first carrier 132 is embodied as a generally open structure that receives the first fluid tube 134 therein. Further, the first carrier 132 also receives a portion of the second fluid tube 136 therein. The first carrier 132 is arcuate in shape, such that the first carrier 132 may be accommodated at a corner of the instillation dock 110. Further, the first fluid tube 134 and the second fluid tube 136 are in fluid communication with each other. The first and second fluid tubes 134, 136 may include flexible tubes. The first instillation tube-set 128 further includes a first instillation tube 124 configured to provide fluid communication between the instillation fluid reservoir 120 and the first fluid tube 134. The instillation fluid 122 is directed towards the first fluid tube 134 via the first instillation tube 124.

Further, the instillation dock 110 includes a pump 138 configured to direct the instillation fluid 122 (see FIG. 1) to the wound site 12 (see FIG. 1). In the illustrated embodiments of FIGS. 3 and 4, the pump 138 is a peristaltic pump. Alternatively, the pump 138 may include any fluid pumping device known in the art. The pump 138 is at least partially received within the first carrier 132. The pump 138 is configured to contact the first fluid tube 134 in order to pressurize and deliver the instillation fluid 122 towards the second fluid tube 136.

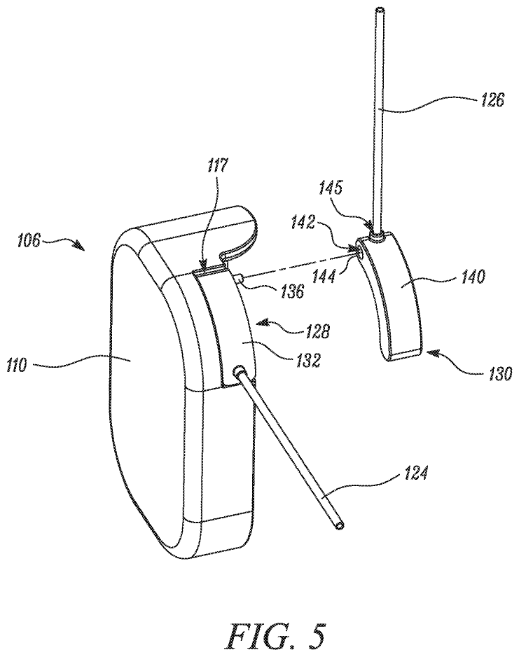

Referring now to FIG. 5, the second instillation tube-set 130 includes a second carrier 140. The second carrier 140 defines an inlet 142 and an outlet 145 in fluid communication with the inlet 142. The second carrier 140 is arcuate in shape and is substantially similar in shape and size to the first carrier 132. The second instillation tube-set 130 further includes a valve 144 disposed at the inlet 142 of the second carrier 140. The valve 144 is biased in a normally closed position. The valve 144 may allow passage and receipt of the instillation fluid 122 within the second carrier 140. Specifically, when the second instillation tube-set 130 is coupled with the first instillation tube-set 128, the valve 144 moves to an open position. Accordingly, the instillation fluid 122 may be directed towards and received within the second carrier 140. Further, when the second instillation tube-set 130 is detached from the first instillation tube-set 128, the valve 144 moves to the closed position, thereby preventing passage of the instillation fluid 122 towards the second instillation tube-set 130. The second instillation tube-set 130 further includes a second instillation tube 126 configured to provide fluid communication between the second carrier 140 and the wound site 12 (see FIG. 1). The instillation fluid 122 received in the second carrier 140 is directed towards the wound site 12 via the second instillation tube 126.

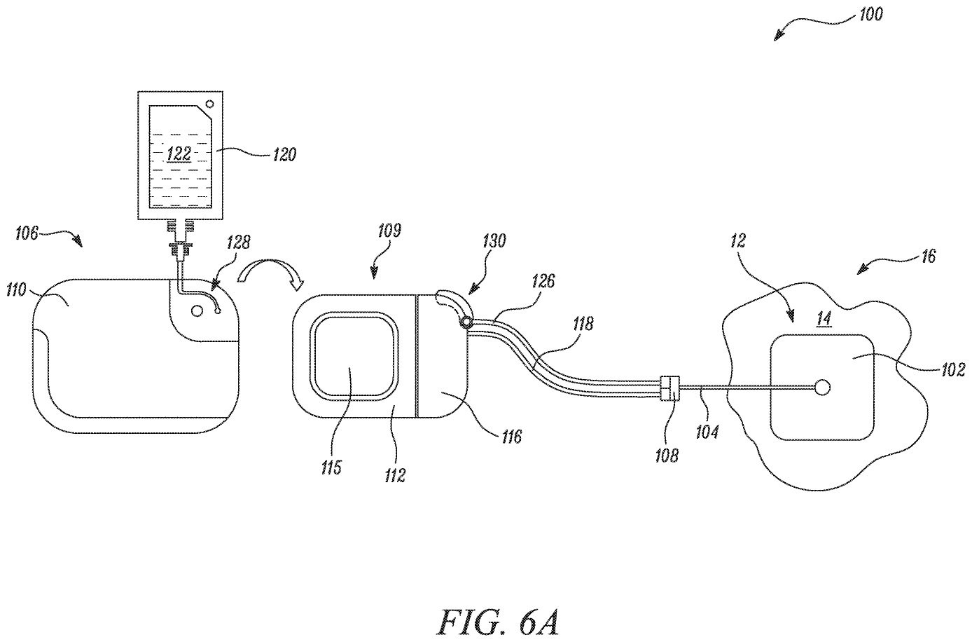

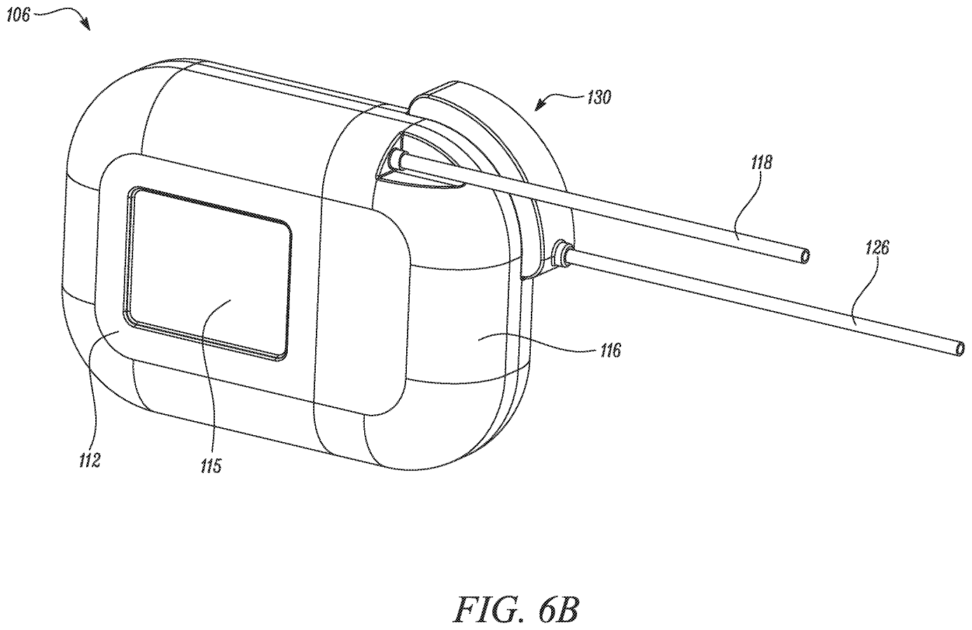

FIG. 6A illustrates a schematic view of the NPWT system 100, according to an embodiment of the present disclosure. As illustrated in FIG. 6A, the therapy assembly 109 may be detached from the instillation dock 110 based on completion of the fluid instillation therapy. Referring now to FIG. 6B, the therapy assembly 109 is illustrated in a detached state. Specifically, the therapy unit 112 and the exudate canister 116 are detached from the instillation dock 110 (see FIG. 6A). In some embodiments, the second instillation tube-set 130 may still be coupled with the exudate cannister 116 when the therapy unit 112 and the exudate canister 116 are detached from the instillation dock 110. Further, in the detached state, the exudate canister 116 and the therapy unit 112 apply the negative pressure therapy to the wound site 12. For example, in some cases, when the user 16 may only require the negative pressure therapy at the wound site 12, the NPWT system 100 may be used in the detached state. Thus, in the detached state, the therapy unit 112 and the exudate canister 116 may be embodied as a portable unit that may improve patient mobility while the negative pressure therapy is in progress. Further, when the fluid instillation therapy is required, the exudate canister 116 and the therapy unit 112 may be again coupled to the instillation dock 110.

The NPWT system 100 disclosed herein may be easy to use. The therapy unit 112 and the exudate canister 116 may be used to apply the negative pressure therapy to the wound site 12 even when the therapy unit 112 and the exudate canister 116 are detached from the instillation dock 110. Specifically, when the user 16 needs to move, the therapy unit 112 and the exudate canister 116 may be easily detached from the instillation dock 110 to improve mobility. Further, the therapy unit 112 and the exudate canister 116 may be easily attached and/or detached from the instillation dock 110, thereby improving usability of the NPWT system 100.

FIG. 7 illustrates a block diagram of the NPWT system 100, according to an embodiment of the present disclosure. As shown in FIG. 7, the instillation dock 110 includes a first controller 146. The first controller 146 may be disposed within the instillation dock 110. The first controller 146 is further configured to generate a control signal S1 for operating the pump 138 when each of the therapy unit 112, the first instillation tube-set 128, and the second instillation tube-set 130 is coupled with the instillation dock 110. The control signal S1 activates the pump 138 in order to deliver the instillation fluid 122 (see FIG. 1) to the wound site 12 (see FIG. 1). Further, the therapy unit 112 includes a second controller 150. The second controller 150 may be disposed within the therapy unit 112.

The first controller 146 is configured to monitor a coupling of each of the therapy unit 112, the exudate canister 116 (see FIG. 1), the first instillation tube-set 128 (shown in FIG. 2), and the second instillation tube-set 130 (see FIG. 2) with the instillation dock 110. Specifically, the first controller 146 monitors efficacy of connection between the therapy unit 112, the first instillation tube-set 128, and the second instillation tube-set 130 with the instillation dock 110. Only when the components of the NPWT system 100 are connected in a suitable manner, the first controller 146 generates the control signal S1 for operating the pump 138. Specifically, the first controller 146 may determine if the therapy unit 112 is mechanically coupled with the instillation dock 110. For this purpose, at least one of the therapy unit 112 and the instillation dock 110 includes a sensor 148. In the illustrated embodiment of FIG. 7, the therapy unit 112 includes the sensor 148. Alternatively, the instillation dock 110 may include the sensor 148. When the therapy unit 112 is coupled with the instillation dock 110, the sensor 148 is configured to generate a signal S2 indicative of the coupling of the therapy unit 112 with the instillation dock 110. In some examples, the sensor 148 may be a switch or any other device that may confirm a mechanical or electrical connection of the therapy unit 112 with the instillation dock 110. In some examples, the sensor 148 may be in direct communication with the first controller 146. In other examples, the sensor 148 may be in communication with the first controller 146 via the second controller 150. Further, the first controller 146 receives the signal S2 from the sensor 148, via the second controller 150. Upon receiving the signal S2, the first controller 146 may generate the control signal S1 for operating the pump 138 in order to apply the fluid instillation therapy to the wound site 12.

Further, the first controller 146 may determine if the first instillation tube-set 128 is mechanically coupled with the instillation dock 110. In an example, the NPWT system 100 may include a sensor that detects the mechanical coupling of the first instillation tube-set with the instillation dock 110. The first controller 146 may also determine if the first instillation tube-set 128 is in a fluid-tight coupling with the second instillation tube-set 130. In an example, the NPWT system 100 may include a sensor that detects the fluid-tight coupling of the first instillation tube-set 128 with the second instillation tube-set 130.

The first controller 146 is further configured to generate a first alert if any one of the therapy unit 112, the first instillation tube-set 128, and the second instillation tube-set 130 is detached from the instillation dock 110. Specifically, when the components of the NPWT system 100 are not connected in a suitable manner, the first controller 146 generates the first alert for alerting the user 16 or a medical personnel to verify if the components of the NPWT system 100 have been suitably connected with each other. The first alert may include an audio notification or a visual notification. For example, the instillation dock 110 may include a speaker to generate an audible message, or a sound, such as a beep, to alert the user 16 or the medical personnel regarding incorrect connections. Alternatively, the instillation dock 110 may include a display screen that may display a text message or an icon to alert the user 16 or the medical personnel regarding incorrect connections. In other example, the first alert may include a combination of the audio notification and the visual notification. In some examples, the first alert may include flashing of a light or a haptic feedback. Thus, the NPWT system 100 of the present disclosure may monitor the efficacy of connections between the instillation dock 110 and other components of the NPWT system 100 and also provides the first alert, thereby reducing any events of inaccurate fluid instillation therapy.

Further, in an example, the second controller 146 may determine if the therapy unit 112 is mechanically coupled with the exudate canister 116. In an example, the NPWT system 100 may include a sensor that detects the mechanical coupling of the therapy unit 112 with the exudate canister 116. Furthermore, the second controller 146 may also determine if the therapy unit 112 is in a fluid-tight coupling with the exudate canister 116. In an example, the NPWT system 100 may include a sensor that detects the fluid-tight coupling of the therapy unit 112 with the exudate canister 116.

Further, the second controller 150 is configured to generate a second alert to notify the user 16 or the medical personnel regarding an impending fluid instillation therapy to the wound site 12. Based on the generation of the second alert, the user 16 or the medical personnel may couple the therapy unit 112 and the exudate canister 116 with the instillation dock 110 for commencing the fluid instillation therapy. The second controller 150 is further configured to generate a third alert to notify the user 16 regarding completion of the fluid instillation therapy to the wound site 12. Upon generation of the third alert, each of the exudate canister 116 and the therapy unit 112 may be detachable from the instillation dock 110. Upon being detached from the instillation dock 110, the exudate canister 116 and the therapy unit 112 may be still configured to apply the negative pressure therapy to the wound site 12. Thus, the negative pressure therapy can be applied at the wound site 12 even when the exudate canister 116 and the therapy unit 112 are detached from the instillation dock 110. The second and third alerts may include an audio notification or a visual notification. For example, the therapy unit 112 may include a speaker to generate an audible message, or a sound, such as a beep, to alert the user 16 or the medical personnel. Alternatively, the therapy unit 112 may include a display screen that may display a text message or an icon to alert the user 16 or the medical personnel. In other examples, the second and third alerts may include a combination of the audio notification and the visual notification. In some examples, the second and third alerts may include flashing of a light or a haptic feedback.

Thus, the NPWT system 100 of the present disclosure may monitor efficacy of connections between the instillation dock 110 and other components of the NPWT system 100 and also provides the second and third alerts, thereby reducing any events of inaccurate negative pressure therapy. Further, the NPWT system 100 may be cost-effective as well as portable and compact in size. Thus, the NPWT system 100 may be easy to handle by users/any medical personnel.

It should be noted that the first and second controllers 146, 150 may include one or more memories (e.g., a non-transitory computer readable medium) and one or more processors communicably coupled with the one or more memories. Each memory may include, for example, a flash memory, a random-access memory (RAM), and an electrically erasable programmable read-only memory (EEPROM). The memories may store data, such as, algorithms, instructions, and/or arithmetic operations. The first and second controllers 146, 150 may execute various types of digitally stored instructions, such as, a software or an algorithm, retrieved from the memories, and/or a firmware program which may enable the first and second controllers 146, 150 to perform a wide variety of operations. Although aspects of the present disclosure may be described generally as being stored in the memories, it may be contemplated that these aspects may be stored on, and read from, different types of computer program products or computer-readable media such as hard disks, floppy disks, optical media, compact disc-read only memory (CD-ROM), or other forms of RAM or read only memory (ROM).

Moreover, the processors may be any device that executes code and perform logical operations. It should be noted that each processor may embody a single microprocessor or multiple microprocessors for receiving various input signals. Numerous commercially available microprocessors may be configured to perform the functions of the processors. Each processor may further include a general processor, a central processing unit, an application specific integrated circuit (ASIC), a digital signal processor, a field programmable gate array (FPGA), a digital circuit, an analog circuit, a controller, a microcontroller, any other type of processor, or any combination thereof. Each processor may include one or more components that may be operable to execute computer executable instructions or computer code that may be stored and retrieved from the memories.

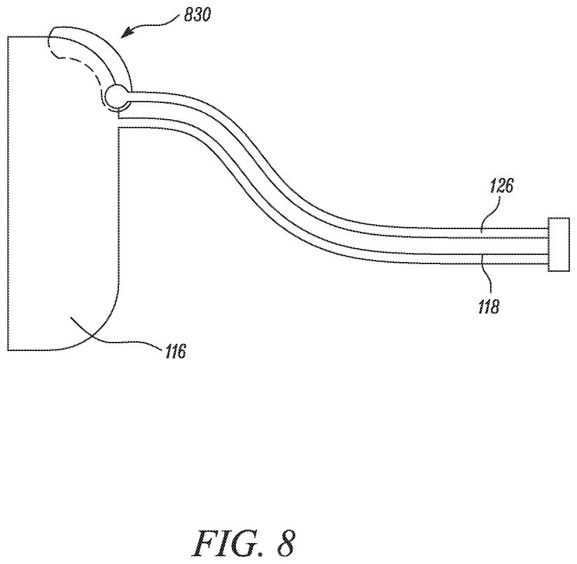

FIG. 8 illustrates a schematic view of a second instillation tube-set 830, according to another embodiment of the present disclosure. In the illustrated embodiment of FIG. 8, the second instillation tube-set 830 is integral with the exudate canister 116. For example, the second instillation tube-set 830 may be integrally coupled to a rear side of the exudate canister 116. In other embodiments, it may be contemplated that the second instillation tube-set 830 is disposed within the exudate canister 116. For example, the exudate canister 116 may include a separate compartment for receipt and direction of the instillation fluid 122 (see FIG. 1). In such embodiments, the exudate canister 116 may include two separate fluid paths. Specifically, the exudate canister 116 may include a first fluid path for the negative pressure therapy and a second fluid path for the fluid instillation therapy.

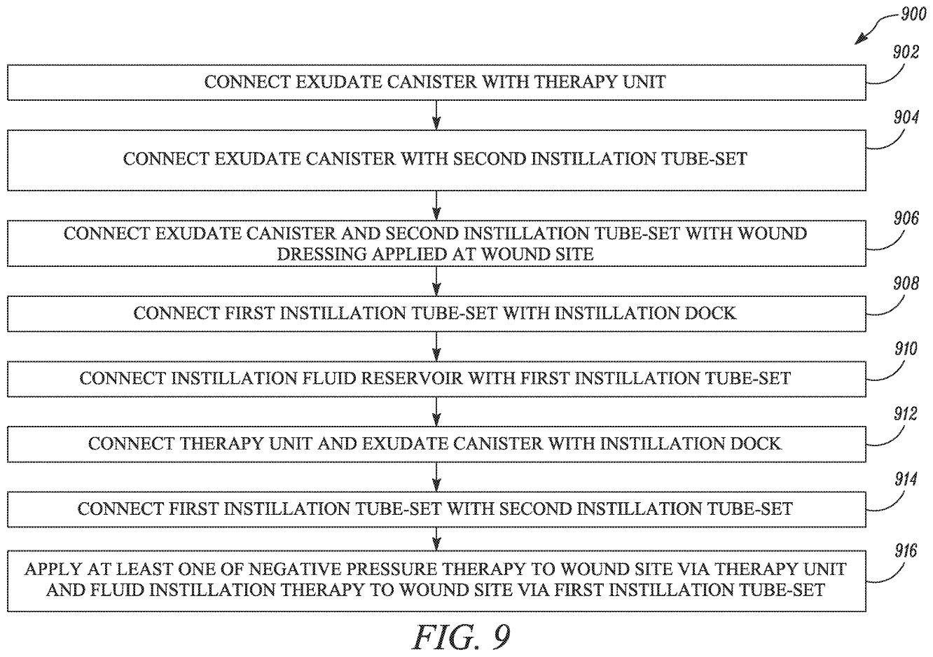

FIG. 9 illustrates a flowchart depicting a method 900 of using the NPWT system 100. The method 900 will now be explained in detail with reference to FIGS. 1-7, and 9.

Referring to FIGS. 1-7, and 9, at step 902, the exudate canister 116 is connected with the therapy unit 112. At step 904, the exudate canister 116 is connected with the second instillation tube-set 130. At step 906, the exudate canister 116 and the second instillation tube-set 130 are connected with the wound dressing 102 applied at the wound site 12. At step 908, the first instillation tube-set 128 is connected with the instillation dock 110. At step 910, the instillation fluid reservoir 120 is connected with the first instillation tube-set 128. At step 912, the therapy unit 112 and the exudate canister 116 are connected with the instillation dock 110.

At step 914, the first instillation tube-set 128 is connected with the second instillation tube-set 130. At step 916, at least one of the negative pressure therapy is applied to the wound site 12 via the therapy unit 112 and the fluid instillation therapy is applied to the wound site 12 via the first instillation tube-set 128.

In some embodiments, the pump 138 of the instillation dock 110 is operated. Further, the fluid instillation therapy is applied to the wound site 12 based on the operation of the pump 138.

In some embodiments, the therapy unit 112 and the exudate canister 116 are detached from the instillation dock 110. Further, the negative pressure source 114 is operated. Moreover, the negative pressure therapy is applied to the wound site 12 based on the operation of the negative pressure source 114 while the therapy unit 112 and the exudate canister 116 are detached from the instillation dock 110.

Unless otherwise indicated, all numbers expressing feature sizes, amounts, and physical properties used in the specification and claims are to be understood as being modified by the term “about”. Accordingly, unless indicated to the contrary, the numerical parameters set forth in the foregoing specification and attached claims are approximations that can vary depending upon the desired properties sought to be obtained by those skilled in the art utilizing the teachings disclosed herein.

Although specific embodiments have been illustrated and described herein, it will be appreciated by those of ordinary skill in the art that a variety of alternate and/or equivalent implementations can be substituted for the specific embodiments shown and described without departing from the scope of the present disclosure. This application is intended to cover any adaptations or variations of the specific embodiments discussed herein. Therefore, it is intended that this disclosure be limited only by the claims and the equivalents thereof.

1. A negative pressure wound therapy (NPWT) system for treatment of a wound site, the NPWT system comprising:
an instillation dock comprising:
a pump configured to direct an instillation fluid to the wound site, and
a port configured to receive a first instillation tube-set and facilitate engagement between the pump and the first instillation tube-set, wherein the first instillation tube-set is adapted to transport the instillation fluid, and wherein the port is configured such that, during operation, the instillation fluid is fluidly isolated from the port; and
a therapy unit adapted to be removably coupled with the instillation dock, the therapy unit comprising a negative pressure source and an exudate canister, wherein the negative pressure source is configured to provide negative pressure to the wound site.

2. The NPWT system of claim 1 further comprising a second instillation tube-set configured to be coupled with the instillation dock, wherein the second instillation tube-set is configured to fluidly communicate the first instillation tube-set and the wound site.

3. The NPWT system of claim 2, wherein the second instillation tube-set is removably coupled to the exudate canister.

4. The NPWT system of claim 2, wherein the second instillation tube-set is integral with the exudate canister.

5. The NPWT system of claim 2, wherein the fluid instillation assembly further comprises an instillation fluid reservoir configured to contain the instillation fluid therein, and wherein the instillation fluid reservoir is in fluid communication with the wound site via the first instillation tube-set and the second instillation tube-set.

6. The NPWT system of claim 5, wherein the first instillation tube-set comprises:
a first carrier comprising a first fluid tube and a second fluid tube; and
a first instillation tube configured to provide fluid communication between the instillation fluid reservoir and the first fluid tube.

7. The NPWT system of claim 6, wherein the pump is at least partially received within the first carrier.

8. The NPWT system of claim 2, wherein the instillation dock further comprises a first controller, and wherein the first controller is configured to:
monitor a coupling of each of the therapy unit, the first instillation tube-set, and the second instillation tube-set with the instillation dock; and
generate a control signal for operating the pump when each of the therapy unit, the first instillation tube-set, and the second instillation tube-set is coupled with the instillation dock.

9. The NPWT system of claim 8, wherein the first controller is further configured to generate a first alert if any one of the therapy unit, the first instillation tube-set, and the second instillation tube-set is detached from the instillation dock.

10. The NPWT system of claim 8, wherein at least one of the therapy unit and the instillation dock comprises a sensor, and wherein, when the therapy unit is coupled with the instillation dock, the sensor is configured to generate a signal indicative of a coupling of the therapy unit with the instillation dock.

11. The NPWT system of claim 2, wherein the second instillation tube-set comprises:
a second carrier defining an inlet and an outlet in fluid communication with the inlet; and
a second instillation tube configured to provide fluid communication between the second carrier and the wound site.

12. The NPWT system of claim 11, wherein the second instillation tube-set further comprises a valve disposed at the inlet of the second carrier, and wherein the valve is biased in a normally closed position.

13. The NPWT system of claim 1, wherein the exudate canister comprises an exudate tube, and wherein the exudate tube provides fluid communication between the exudate canister and the wound site.

14. The NPWT system of claim 1, wherein the therapy unit comprises a second controller, and wherein the second controller is configured to generate a second alert to notify a user regarding an impending fluid instillation therapy to the wound site.

15. The NPWT system of claim 14, wherein the second controller is further configured to generate a third alert to notify the user regarding completion of a fluid instillation therapy to the wound site.

16. The NPWT system of claim 15, wherein, upon generation of the third alert, each of the exudate canister and the therapy unit is detachable from the instillation dock, and wherein, upon being detached from the instillation dock, the exudate canister and the therapy unit are configured to apply a negative pressure therapy to the wound site.

17. A method of using the NPWT system of claim 2, the method comprising:
connecting the exudate canister with the therapy unit;
connecting the exudate canister with the second instillation tube-set;
connecting the exudate canister and the second instillation tube-set with a wound dressing applied at the wound site;
connecting the first instillation tube-set with the instillation dock;
connecting an instillation fluid reservoir with the first instillation tube-set;
connecting the therapy unit and the exudate canister with the instillation dock;
connecting the first instillation tube-set with the second instillation tube-set; and
applying at least one of:
a negative pressure therapy to the wound site via the therapy unit; and
a fluid instillation therapy to the wound site via the first instillation tube-set.

18. The method of claim 17, further comprising:
operating the pump of the instillation dock; and
applying the fluid instillation therapy to the wound site based on the operation of the pump.

19. The method of claim 17, further comprising:
detaching the therapy unit and the exudate canister from the instillation dock;
operating the negative pressure source; and
applying the negative pressure therapy to the wound site based on the operation of the negative pressure source while the therapy unit and the exudate canister are detached from the instillation dock.
